(19) United States
(12) Patent Application Publication
(10) Pub. No.: US 2019/0074077 A1
(43) Pub. Date: Mar. 7, 2019

(54) PRESCRIPTION MANAGEMENT SYSTEM AND METHOD

(71) Applicant: TAIWAN FINTECH CORPORATION, FUSION$360, TAIPEI CITY (TW)

(72) Inventors: Yen-Hung KUO, TAIPEI CITY (TW); Ya-Hui CHAN, TAIPEI CITY (TW); Yi-An CHEN, TAIPEI CITY (TW); Yen-Cheng KUNG, TAIPEI CITY (TW); Chang-Hsien CHUANG, TAIPEI CITY (TW)

(21) Appl. No.: 15/898,658

(22) Filed: Feb. 18, 2018

(30) Foreign Application Priority Data
Sep. 4, 2017 (TW) 106130136

(51) Int. Cl. G16H 20/10; G16H 10/60

(52) U.S. Cl. CPC G16H 20/10; G06Q 10/10; G16H 10/60

(57) ABSTRACT

A prescription management system and method are provided. The prescription management system: (a) calculates at least one insurance fee and an out-of-pocket fee of a prescription based on at least one medicine name and a medicine price database, (b) calculates an expiration date of the prescription according to a prescription date and an allowed number of days, (c) records an identity code of the prescription, the prescription date, a user identity, the at least one medicine name, the expiration date, the at least one insurance fee, and the out-of-pocket fee of the prescription into a historical prescription ledger, and (d) records a prescription filled date, the identity code of the prescription, at least one filled medicine name, the at least one insurance fee, and the out-of-pocket fee into an insurance payment ledger.

### PRIORITY

This application claims priority to Taiwan Patent Application No. 106130136 filed on Sep. 4, 2017, which is hereby incorporated by reference in its entirety.

### FIELD

The present invention relates to a prescription management system and method. More particularly, the present invention relates to a prescription management system and method for automatically clearing fees.

### BACKGROUND

With the development of the medical technology and the aging of the population, the frequency that people visit medical institutions and pharmacies is increasing year by year. Due to the shortage of manpower of the medical institutions and the complicated procedures for medical diagnosis and medicine filling, both the time that people wait for diagnosis and working hours of medical workers are increased, thereby lowering the overall medical quality. Therefore, it is an important issue to improve the operation mode of medical systems in order to increase the medical quality.

Several obvious problems exist in the current medical systems. For example, prescription prescribing, medicine filling, fee paying, and insurance-payment applying involve several different institutions (e.g., the bureau of National Health Insurance, pharmacies, insurance institutions, medical institutions or the like), but a perfect integration mechanism is unavailable between these institutions and, thereby, causing difficulty in data integration and calculation of cash flows. Additionally, a person may visit different medical institutions for diagnosis within a short period of time and get same medicines or medicines having interaction with other medicine because a physician is unable to know other prescriptions prescribed by other physicians for the same person. Furthermore, a filling prompt mechanism is unavailable in current medical systems, so users may forget to fill the medicines in time and/or the prescription may expire. Moreover, the current medical systems cannot trace medicines that have been returned by people or medicines that have not been taken by the people for reference of physicians.

Accordingly, there is an urgent need for a prescription management technology that integrates the prescription writing, medicine filling, the fee clearing, etc.

### SUMMARY

The disclosure includes a prescription management system. The prescription management system comprises a storage, a receiving interface, and a processor, wherein the processor is electrically connected to the storage and the receiving interface. The storage stores a historical prescription ledger and an insurance payment ledger. The receiving interface receives a confirmation signal of prescribing a prescription for a user, wherein the prescription comprises at least one medicine name. The processor calculates at least one insurance fee and an out-of-pocket fee of the prescription based on the at least one medicine name and a medicine price database, calculates an expiration date of the prescription according to a prescription date and an allowed number of days of the prescription, and records an identity code of the prescription, the prescription date, a user identity of the user, the at least one medicine name, the expiration date, the at least one insurance fee, and the out-of-pocket fee of the prescription into the historical prescription ledger. The processor further records a prescription filled date, the identity code of the prescription, at least one filled medicine name, the at least one insurance fee, and the out-of-pocket fee into the insurance payment ledger in response to a filling confirmation signal, wherein the at least one filled medicine name corresponds to the at least one medicine name.

The disclosure also includes a prescription management method, which is adapted for a server system. The server system stores a historical prescription ledger and an insurance payment ledger. The prescription management method comprises the following steps of: (a) receiving a confirmation signal of prescribing a prescription for a user, wherein the prescription comprises at least one medicine name, (b) calculating at least one insurance fee and an out-of-pocket fee of the prescription based on the at least one medicine name and a medicine price database, (c) calculating an expiration date of the prescription according to a prescription date and an allowed number of days of the prescription, (d) recording an identity code of the prescription, the prescription date, a user identity of the user, the at least one medicine name, the expiration date, the at least one insurance fee, and the out-of-pocket fee of the prescription into the historical prescription ledger, and (e) recording a prescription filled date, the identity code of the prescription, at least one filled medicine name, the at least one insurance fee, and the out-of-pocket fee into the insurance payment ledger in response to a filling confirmation signal, wherein the at least one filled medicine name corresponds to the at least one medicine name.

The prescription management mechanism (including the system and the method) adopts at least two ledgers, i.e., a historical prescription ledger and an insurance payment ledger. After receiving a confirmation signal of prescribing a prescription for a user, the prescription management mechanism calculates fees (i.e., an insurance fee and an out-of-pocket fee) and dates relevant to the prescription and then updates the historical prescription ledger accordingly. Moreover, after receiving a filling confirmation signal of the prescription, the prescription management mechanism also updates the insurance payment ledger accordingly. By automatically updating the historical prescription ledger and the insurance payment ledger, the difficulty of data integration and calculation of cash flows in the conventional medical system is overcome. Additionally, the aforesaid historical prescription ledger and the insurance payment ledger may be realized by a blockchain architecture. By the blockchain architecture, information recorded in the historical prescription ledger and the insurance payment ledger can be verified in real time, are traceable, and cannot be tampered. Thereby, a shared prescription management mechanism of high privacy, high efficiency, and high security is provided.

The detailed technology and preferred embodiments implemented for the subject invention are described in the following paragraphs accompanying the appended drawings for people skilled in this field to well appreciate the features of the claimed invention.

### BRIEF DESCRIPTION OF THE DRAWINGS

FIG. 1 is a schematic view illustrating the architecture of a first embodiment of the present invention;

FIG. 2 is a schematic view illustrating the architecture of a second embodiment of the present invention; and

FIG. 3A and FIG. 3B are flowchart diagrams of a third embodiment of the present invention.

### DETAILED DESCRIPTION

In the following descriptions, the prescription management system and method according to certain embodiments of the present invention will be explained with reference to example embodiments thereof. However, these example embodiments are not intended to limit the present invention to any specific example, embodiment, environment, applications, or implementations described in these example embodiments. Therefore, descriptions of these example embodiments are only for purpose of illustration rather than to limit the scope of the present invention.

It shall be appreciated that, in the following embodiments and the attached drawings, elements unrelated to the present invention are omitted from depiction; dimensions of individual elements and dimensional relationships among the individual elements in the attached drawings are only for illustration, but not to limit the scope of the present invention.

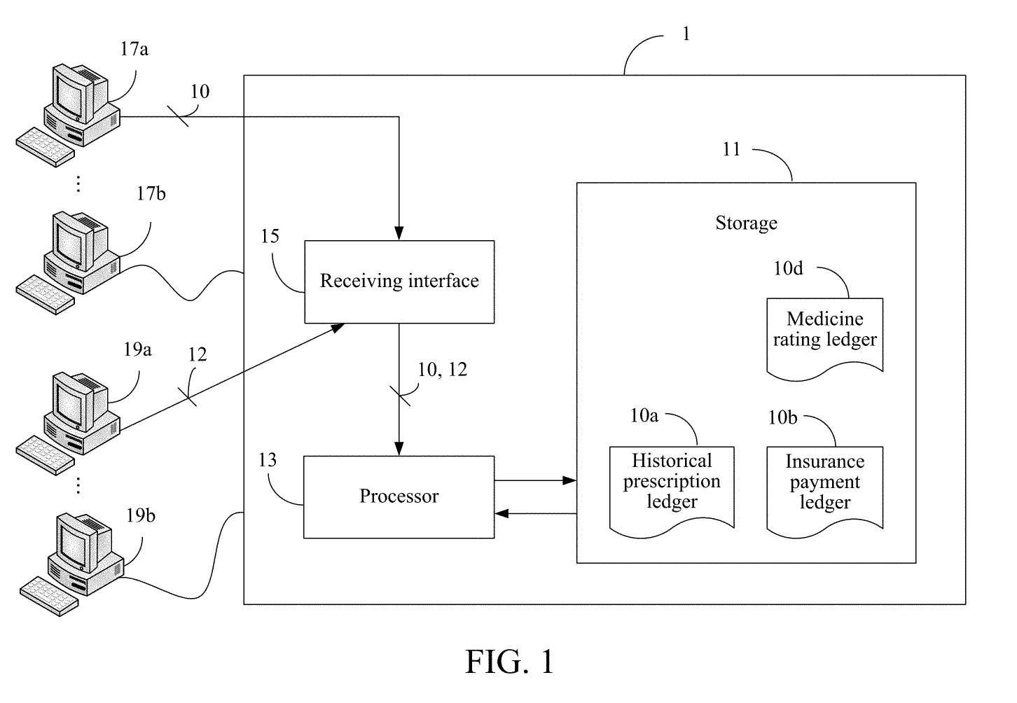

A first embodiment of the present invention is a prescription management system 1 and a schematic view of which is depicted in FIG. 1. The prescription management system 1 comprises a storage 11, a processor 13, and a receiving interface 15, wherein the processor 13 is electrically connected to the storage 11 and the receiving interface 15. The storage 11 may be a memory, a universal serial bus (USB) disk, a hard disk, a mobile disk, a database, or any other storage medium or circuit with the same function and well known to those of ordinary skill in the art. The processor 13 may be any of various processors, central processing units (CPUs), microprocessors, or other computing devices well known to those of ordinary skill in the art. The receiving interface 15 may be various devices capable of receiving signals and data.

The prescription management system 1 is used with a plurality of medical information devices 17a, . . . , 17b and a plurality of pharmacy information devices 19a, . . . , 19b. Each of the medical information devices 17a, . . . , 17b may be a computer, an electronic computing device, or other similar information devices disposed in medical institutions. Each of the pharmacy information devices 19a, . . . , 19b may be a computer, an electronic computing device, or other similar information devices disposed in pharmacies or other similar institutions. Physicians, pharmacists, or users (e.g., patients) using the prescription management system 1 has to register to the prescription management system 1. Please note that the way to register to the prescription management system 1 is not the focus of the present invention and, thus, will not be described herein.

The storage 11 stores a historical prescription ledger 10a and an insurance payment ledger 10b. Generally speaking, the historical prescription ledger 10a records prescriptions that have been prescribed to any user registered to the prescription management system 1 by any physician registered to the prescription management system 1, and while the insurance payment ledger 10b records bill information of each of the prescriptions. It shall be appreciated that, in some embodiments, the historical prescription ledger 10a and the insurance payment ledger 10b may be stored in the storage 11 individually in the form of a blockchain.

In some embodiments, the physician may operate on any of the medical information devices 17a, . . . , 17b to prescribe the prescription. The prescription management system 1 automatically clears relevant fees of the prescription (i.e., calculates an insurance fee and an out-of-pocket fee) after a physician confirms that he/she has prescribed a prescription for a user. Specifically, after a physician has prescribed a prescription for a user, the receiving interface 15 receives a confirmation signal 10 of prescribing the prescription for the user by the physician (e.g., receives from the medical information device 17a). For example, the confirmation signal 10 may comprise a user identity of the user, at least one medicine name comprised in the prescription, a prescription date, and an identity code of the prescription.

After the receiving interface 15 receives the confirmation signal 10, the processor 13 calculates at least one insurance fee and an out-of-pocket fee of the prescription based on the at least one medicine name of the prescription and a medicine price database. Specifically, the medicine price database records a plurality of medicine names and an insurance payment amount corresponding to each of the medicine names. It shall be appreciated that, in some embodiments, the insurance payment amount recorded in the medicine price database is subsidized by the government. The processor 13 calculates an insurance fee of the prescription by querying the medicine price database according to the at least one medicine name of the prescription. The processor 13 may further query a user insurance database according to the user identity of the user to determine whether the user has an unexpired and available insurance (i.e., determine whether the user has an insurance that is unexpired and capable of paying the amount of money corresponding to the at least one medicine name). If the user has an unexpired and available insurance, the processor 13 calculates another insurance fee of the user according to insurance rules of the user recorded in the user insurance database. After calculating all the insurance fees of the user, the processor 13 then calculates the out-of-pocket fee of the user according to all the insurance fees (i.e., the out-of-pocket fee is obtained by subtracting all the insurance fees from the total fee of the prescription).

Additionally, the processor 13 calculates an expiration date of the prescription according to a prescription date and an allowed number of days of the prescription. The allowed number of days may be a preset value, while the expiration date represents the deadline that the user can get the medicine according to the prescription.

Thereafter, the processor 13 records an identity code of the prescription, the prescription date, the user identity of the user, the at least one medicine name, the expiration date, the at least one insurance fee, and the out-of-pocket fee of the prescription into the historical prescription ledger 10a. It shall be appreciated that, in some embodiments, the processor 13 may also record medical relevant information of the user (e.g., the name, age, and diagnostic content of the user, the name of the physician, the name, address, and contact number of the medical institution, the name and the type of the medicine, the content per unit of the medicine, the number and dosage of the medicine, and the medicine use instructions) into the historical prescription ledger 10a.

After the physician prescribes the prescription for the user, the user can get the medicine at the pharmacy. After the user gets the medicine, a pharmacist in the pharmacy transmits a filling confirmation signal 12 to the prescription management system 1 via a pharmacy information device (e.g., the pharmacy information device 19a). The processor 13 records a prescription filled date, the identity code of the prescription, at least one filled medicine name, the at least one insurance fee, and the out-of-pocket fee into the insurance payment ledger 10b in response to the filling confirmation signal 12. The aforesaid at least one filled medicine name is the name of the medicine that the user actually gets and it corresponds to the at least one medicine name of the prescription.

It shall be appreciated that, in some embodiments, the prescription may be a continuous prescription (i.e., the user can get the medicine for several times according to the prescription). In those embodiments, the processor 13 calculates a plurality of filling reminder dates according to the prescription date, at least one number of filling days (i.e., the number of days of the medicine got by the user each time, which may be the same as or different from each other) and an allowed filling duration (i.e., the number of days for the user to get the medicine again before the date when the medicine got by the user last time is used up). The processor further records the filling reminder dates into the historical prescription ledger 10a. In some embodiments, the prescription management system 1 may transmit a filling permission prompt to the user at the beginning of the filling reminder dates. For ease of understanding, a specific example is given herein to show the details that the prescription management system 1 manages the continuous prescription. Herein, it is assumed that the number of filling days of the prescription is 28 (i.e., medicines that can be used for 28 days can be got by the user each time) and the allowed filling duration of the prescription is 10 days. The processor 13 calculates a use-up date of the medicine got by the user for the first time according to the prescription date and the number of filling days. The processor 13 sets the first filling reminder date to be the date that is the allowed filling duration before the use-up date. The processor 13 may set a second filling reminder date to be the date that is the number of filling days after the first filling reminder date, may set a third filling reminder date to be the date that is the number of filling days after the second filling reminder date, and so on.

It shall be appreciated that, in some embodiments, the processor 13 further determines a medicine compounding service charge according to a category of the prescription after the pharmacist in the pharmacy transmits the filling confirmation signal 12 to the prescription management system 1 via the pharmacy information device. The aforesaid category may be a general prescription or a continuous prescription, and the aforesaid medicine compounding service charge is the charge that the government has to pay to the pharmacist.

In some embodiments, the prescription management system 1 further provides a medicine conflict evaluation mechanism for a physician when the physician is going to prescribe a prescription for a user. Specifically, the historical prescription ledger 10a stores a plurality of prescription records, wherein each prescription record is the content of a prescription that has been prescribed to any user registered to the prescription management system 1 by any physician registered to the prescription management system 1. When the physician is going to prescribe a prescription for a user, the processor 13 retrieves at least one unexpired prescription record of the user according to the user identity of the user and a use-up date of each of the prescriptions (i.e., the date when the medicine got by the user according to the prescription will be used up). The processor 13 further determines a current medicine ingredient list according to the at least one medicine name included in the at least one unexpired prescription record and a medicine ingredient database (which records medicine ingredients comprised in different medicine names). For example, the fields of the current medicine ingredient list may comprise medicine name and medicine ingredient. Next, the processor 13 determines a prescription medicine ingredient list according to the at least one medicine name included in the prescription that the physician is going to prescribe and the medicine ingredient database. Then, the processor 13 determines a medicine conflict evaluation result by comparing the current medicine ingredient list with the prescription medicine ingredient list. More specifically, if the current medicine ingredient list and the prescription medicine ingredient list have a same medicine ingredient, the medicine conflict evaluation result is that the medicine that the physician is going to prescribe overlaps with the medicine that the user is currently taking. Under such circumstance, the prescription management system 1 may generate a prompt message to inform the physician to determine whether to verbally instruct the patient to take the overlapped medicine that has been prescribed according to the physician's advice instead of prescribing the medicine again.

In some embodiments, the prescription management system 1 provides another medicine conflict evaluation mechanism for a physician when the physician is going to prescribe a prescription for a user. The processor 13 determines a current medicine ingredient list and a prescription medicine ingredient list in the way addressed in previous paragraph. Thereafter, the processor 13 determines a medicine conflict evaluation result according to the current medicine ingredient list, the prescription medicine ingredient list, and a medicine interaction database. For example, the aforesaid medicine interaction database may record interactions between two medicine ingredients when they are used together. More particularly, the processor 13 generates a plurality of combinations by each medicine ingredient in the current medicine ingredient list with each medicine ingredient in the prescription medicine ingredient list and queries the medicine interaction database for each combination. If any of the aforesaid combinations appears in the medicine interaction database, the medicine conflict evaluation result is that the medicine that the physician is going to prescribe interacts with the medicine that the user is currently taking. Under such circumstance, the prescription management system 1 may generate a prompt message to inform the physician to determine whether to prescribe other medicines instead.

In some embodiments, the storage 11 further stores a medicine rating ledger 10d, which records the physicians who prescribe medicine that will cause conflict and/or interaction with other medicine and the related information. As described previously, the prescription management system 1 may provide two medicine conflict evaluation mechanisms and inform the physician of the medicine conflict evaluation result of the prescription prescribed by the physician via a prompt message. The medicine conflict evaluation result may be that a first ingredient of a first medicine included in the prescription conflicts (e.g., overlaps or interacts) with a second ingredient of a second medicine recorded in a specific prescription record among the at least one unexpired prescription record. If the physician insists on prescribing the prescription, the processor 13 records a physician identity of the physician who prescribes the prescription, the prescription date, the identity code of the prescription, the first medicine, the first ingredient, an identity code of the specific prescription record, the second medicine, and the second ingredient into the medicine rating ledger 10d. Please note that the medicine rating ledger 10d may be stored in the storage 11 in the form of a blockchain in some embodiments.

From the above descriptions, the prescription management system 1 of the first embodiment establishes data connection between the medical institutions, the pharmacies, and the insurance institutions. The prescription management system 1 calculates fees (i.e., the insurance fee and the out-of-pocket fee) and dates related to the prescription after receiving a confirmation signal of prescribing a prescription for a user and then updates the historical prescription ledger 10a accordingly. The physician can quickly learn the necessary medical relevant information of some users according to data recorded in the historical prescription ledger 10a and, thereby, both the medical quality and the diagnosis efficiency can be improved. Moreover, the prescription management system 1 can quickly evaluate whether the medicine that the physician is going to prescribe will cause medicine conflict according to the data recorded in the historical prescription ledger 10a and generate a prompt message for the physician and thereby assist the physician in improving the diagnosis accuracy. If the medicine prescribed by the physician causes medicine conflict(s), relevant information will be recorded in the medicine rating ledger 10d so that relevant people (e.g., users, responsible institutions) can refer thereto and/or audit.

The prescription management system 1 also provides a filling prompt mechanism so that the user can go to the pharmacy to get the medicine in due time. With such a mechanism, time waste caused by user going to the pharmacy at an undue time can be avoided and failure of prescription caused by user forgetting to get the medicine can be avoided as well. Such a mechanism can increase the medicine filling rate and, thereby, the number of times of unnecessary repeated diagnosis can be reduced and the medical resources can be utilized properly and fully. Hence, the problem that the medial quality is lowered by the shortage of manpower in the medical institutions can be overcome.

Additionally, in the first embodiment, the prescription management system 1 updates the insurance payment ledger 10b after receiving a filling confirmation signal of a prescription. Through the insurance payment ledge 10b and the historical prescription ledge 10a, the prescription management system 1 effectively integrates cash flows during the process from prescribing the prescription by the physician to getting the medicine by the user. Moreover, the prescription management system 1 can automatically determine a medicine compounding service charge according to a category of the prescription and effectively integrate data of the pharmacies, the medical institutions, the insurance institutions, and the users, thereby solving the medical efficiency issue that has not yet been solved in the prior art.

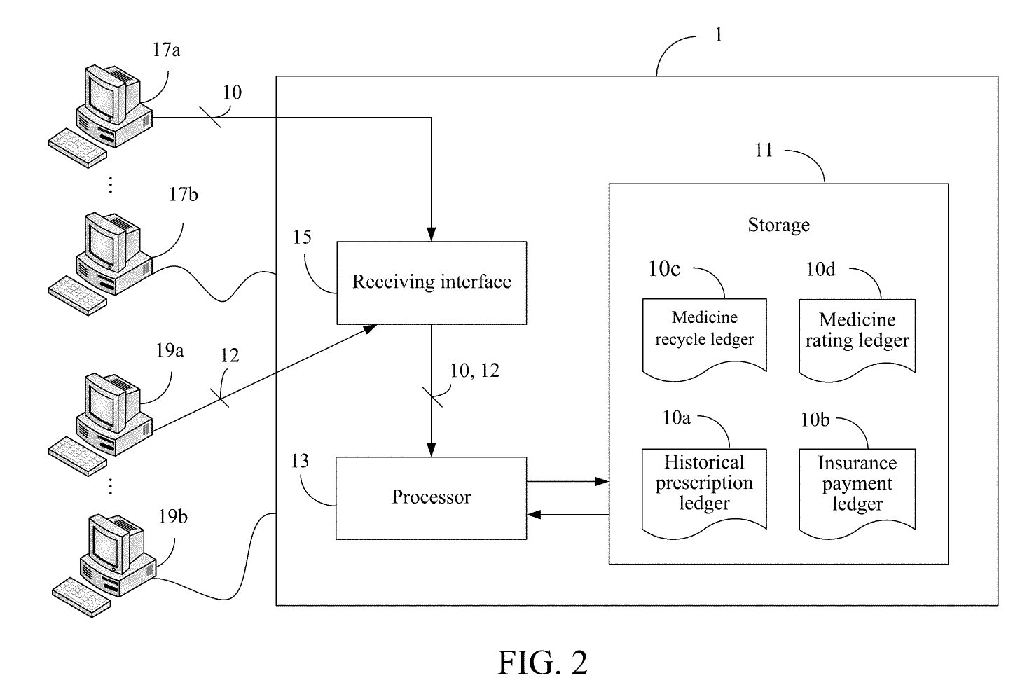

Please refer to FIG. 2 for a second embodiment of the present invention. In this embodiment, the prescription management system 1 can also execute all the operations set forth in the first embodiment, have the same functions, and deliver the same technical effects as the first embodiment, which will not be repeated herein. Hereinafter, only differences between the second embodiment and the first embodiment will be detailed. In this embodiment, the storage 11 further stores a medicine recycle ledger 10c. Generally speaking, the medicine recycle ledger 10c records information of medicines that have not been taken or have been returned by any user registered to the prescription management system 1.

In real life, the user may stop taking the medicine because he/she feels better or because of other factors (e.g., work scheduling, living habits, or religious faith). When those situations happen, the medicine previously got by the user is not used up, or the user does not go to the pharmacy to get the medicine any more according to the filling reminder dates of the continuous prescription. In this embodiment, if the filling confirmation signal of the continuous prescription is not received by the prescription management system 1, the processor 13 automatically records a non-filling message in the medicine recovery ledge 10c, wherein the aforesaid non-filling message comprises the user identity as well as the medicine name of at least one medicine not taken by the user. Additionally, the user may also voluntarily return the medicine that has not been used up by the user (i.e., the prescription management system 1 passively receives remaining medicine information reported by the user). The processor 13 records the user identity as well as the medicine name of at least one medicine returned by the user and a remaining medicine amount corresponding to each of the at least one medicine name into the medicine recycle ledger 10c. In some embodiments, the medicine recycle ledger 10c may be stored in the storage 11 in the form of a blockchain.

In this embodiment, the prescription management system 1 provides a medicine evaluation mechanism for a physician when the physician is going to prescribe a prescription for a user. Specifically, the processor 13 determines a medicine-prescribing prompt by comparing the at least one medicine name corresponding to the user identity of the user in the medicine recycle ledger 10c with the at least one medicine name of the prescription (i.e., determining whether the medicine that the physician is going to prescribe has ever been returned by the user or has ever not be taken by the user according to the filling reminder dates) so that the physician is able to evaluate whether to prescribe the medicine in a reduced amount or whether to prescribe other medicines alternatively.

From the above descriptions, the prescription management system 1 of the second embodiment records the medicine that is not used up or taken by the user due to environmental or personal factors into the medicine recycle ledger 10c and, thereby, provide the physician with another medicine evaluation mechanism. Through the medicine recycle ledger 10c, the physician will know the medicine usage habit of the user taking medicine and then appropriately adjust the prescription that the physician is going to prescribe. Thereby, waste of medicine can be reduced and utilization ratio of medicine can be increased. In this way, the problem that the medicine management efficiency is poor of the prior art can be solved.

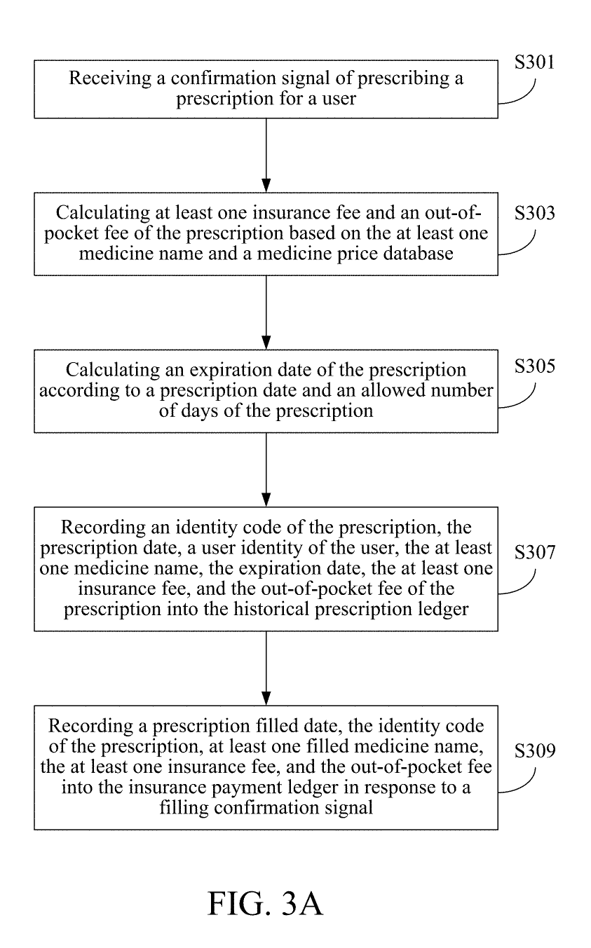

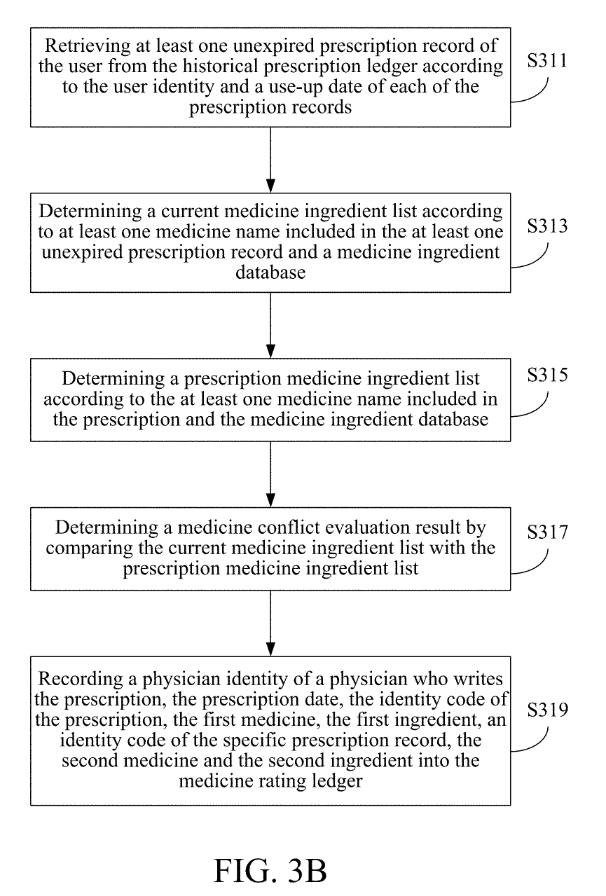

A third embodiment of the present invention is a prescription management method and a flowchart thereof is depicted in FIG. 3A and FIG. 3B. The prescription management method is adapted for a server system, wherein the server system may comprise one server (e.g., the prescription management system 1 of the first embodiment) or several servers. In this embodiment, the server system stores a historical prescription ledger and an insurance payment ledger.

In step S301, the server system receives a confirmation signal of prescribing a prescription for a user, wherein the prescription comprises at least one medicine name. Next, in step S303, the server system calculates at least one insurance fee and an out-of-pocket fee of the prescription based on the at least one medicine name and a medicine price database. In step S305, the server system calculates an expiration date of the prescription according to a prescription date and an allowed number of days of the prescription. In step S307, the server system records an identity code of the prescription, the prescription date, a user identity of the user, the at least one medicine name, the expiration date, the at least one insurance fee, and the out-of-pocket fee of the prescription in the historical prescription ledger. In step S309, the server system records a prescription filled date, the identity code of the prescription, at least one filled medicine name, the at least one insurance fee, and the out-of-pocket fee in the insurance payment ledger in response to a filling confirmation signal. It shall be appreciated that the order for executing the steps S303 and S305 is not limited in the present invention. The steps S303 and S305 may be executed together or separately as longs as the steps S303 and S305 are executed before the step S307.

It shall be appreciated that, in some embodiments, the prescription is a continuous prescription. In these embodiments, the prescription management method may further execute a step to calculate a plurality of filling reminder dates according to the prescription date, a number of filling days, and an allowed filling duration, and the step S307 further records these filling reminder dates in the historical prescription ledger.

It shall be appreciated that, in some embodiments, the prescription management method may further execute a step to determine a medicine compounding service charge according to a category of the prescription (e.g., a general prescription or a continuous prescription), and the step S307 further records the medicine compounding service charge in the historical prescription ledger.

It shall be appreciated that, in some embodiments, the server system further stores a medicine recycle ledger. In those embodiments, the prescription management method may further execute a step to record the user identity, at least one medicine name included in a medicine recycle item of the user, and a remaining medicine amount corresponding to each of the at least one medicine name in the medicine recycle ledger. This typically occurs when the filling confirmation signal of a continuous prescription is not received by the server system or the user voluntarily returns the medicine that has not been used by the user. In those embodiments, the prescription management method may further execute another step in which the server system decides a medicine-prescribing prompt by comparing each of the medicine names of the prescription with the at least one medicine name corresponding to the user identity in the medicine recycle ledger. The physician may refer to the medicine-prescribing prompt when prescribing the prescription.

In some embodiments, the prescription management method may further execute the flowchart shown in FIG. 3B to provide a medicine conflict evaluation mechanism, which assists the physician in prescribing a prescription. In those embodiments, the historical prescription ledger has stored a plurality of prescription records. In step S311, the server system retrieves at least one unexpired prescription record of the user from the historical prescription ledger according to the user identity of the user and a use-up date of each of the prescription records. In step S313, the server system determines a current medicine ingredient list according to at least one medicine name included in the at least one unexpired prescription record and a medicine ingredient database. In step S315, the server system determines a prescription medicine ingredient list according to the at least one medicine name included in the prescription and the medicine ingredient database. It shall be appreciated that the order for executing the steps S313 and S315 is not limited in the present invention. The steps S313 and S315 may be executed simultaneously or separately as longs as the steps S313 and S315 are executed before step S317. In the step S317, the server system determines a medicine conflict evaluation result by comparing the current medicine ingredient list and the prescription medicine ingredient list. For example, if the current medicine ingredient list and the prescription medicine ingredient list have a same medicine ingredient, the medicine conflict evaluation result is that the medicine that the physician is going to prescribe overlaps with the medicine that the user is currently taking.

In some embodiments, the step S317 may executed by the server system to determine a medicine conflict evaluation result according to the current medicine ingredient list, the prescription medicine ingredient list, and a medicine interaction database. For example, the aforesaid medicine interaction database may record interactions between two medicine ingredients when they are used simultaneously. In those embodiments, the step S317 generates a plurality of combinations by each medicine ingredient in the current medicine ingredient list with each medicine ingredient in the prescription medicine ingredient list and queries the medicine interaction database by each combination. If a certain combination among the aforesaid combinations exists in the medicine interaction database, the medicine conflict evaluation result is that the medicine that the physician is going to prescribe interacts with the medicine that the user is currently taking.

In some embodiments, the server system further stores a medicine rating ledger, which records information related to prescribed prescriptions that cause medicine conflicts. In those embodiments, the medicine conflict evaluation result is that a first ingredient of a first medicine included in the prescription conflicts with a second ingredient of a second medicine recorded in a specific prescription record among the at least one unexpired prescription record. The prescription management method further executes step S319, in which the server system records a physician identity of a physician who prescribes the prescription, the prescription date, the identity code of the prescription, the first medicine, the first ingredient, an identity code of the specific prescription record, the second medicine and the second ingredient in the medicine rating ledger.

In addition to the aforesaid steps, the third embodiment can execute all the operations and steps set forth in the first and the second embodiments, have the same functions, and deliver the same technical effects as the first and the second embodiments. How the third embodiment executes these operations and steps, has the same functions, and delivers the same technical effects as the first and the second embodiments will be readily appreciated by those of ordinary skill in the art based on the explanation of the first and the second embodiments, and thus will not be further described herein.

It shall be appreciated that, in the specification of the present invention, terms “first” and “second” used in the first medicine and the second medicine are only used to mean that these medicines are different medicines. Terms “first” and “second” used in the first ingredient and the second ingredient are only used to mean that these ingredients are different ingredients.

According to the above descriptions, the prescription management technology (including the system and the method) of the present invention calculates fees (i.e., the insurance fee and the out-of-pocket fee) and dates relevant to the prescription after a confirmation signal of writing a prescription for a user is received and then updates the historical prescription ledger accordingly. Moreover, the prescription management technology (including the system and the method) of the present invention updates the insurance payment ledger after a filling confirmation signal of the prescription is received. By automatically updating the historical prescription ledger and the insurance payment ledger, data connection between the medical institutions, the pharmacies, and the insurance institutions is established in the present invention to overcome difficulty in data integration and calculation of cash flows in the conventional medical systems. Additionally, the present invention may also adopt the medicine recycle ledger and the medicine rating ledger and provide a plurality of evaluation and prompt mechanisms in cooperation. Through the operations in the aforesaid embodiments, the prescription management technology (including the system and the method) of the present invention integrates data connection and management among different institutions involved in a medical process, thereby overcoming issues that cannot be solved in the prior art such as “improving the medical efficiency” and “medicine prompting periodically” or the like.

The above disclosure is related to the detailed technical contents and inventive features thereof. People skilled in this field may proceed with a variety of modifications and replacements based on the disclosures and suggestions of the invention as described without departing from the characteristics thereof. Nevertheless, although such modifications and replacements are not fully disclosed in the above descriptions, they have substantially been covered in the following claims as appended.

What is claimed is:

1. A prescription management system, comprising:
a storage, being configured to store a historical prescription ledger and an insurance payment ledger;
a receiving interface, being configured to receive a confirmation signal of prescribing a prescription for a user, wherein the prescription comprises at least one medicine name; and
a processor, being electrically connected to the storage and the receiving interface and configured to perform the following operations after the receiving interface receives the confirmation signal: (a) calculating at least one insurance fee and an out-of-pocket fee of the prescription based on the at least one medicine name and a medicine price database, (b) calculating an expiration date of the prescription according to a prescription date and an allowed number of days of the prescription, and (c) recording an identity code of the prescription, the prescription date, a user identity of the user, the at least one medicine name, the expiration date, the at least one insurance fee, and the out-of-pocket fee of the prescription into the historical prescription ledger,
wherein the processor is further configured to record a prescription filled date, the identity code of the prescription, at least one filled medicine name, the at least one insurance fee, and the out-of-pocket fee into the insurance payment ledger in response to a filling confirmation signal,
wherein the at least one filled medicine name corresponds to the at least one medicine name.

2. The prescription management system of claim 1, wherein the prescription is a continuous prescription, the processor further calculates a plurality of filling reminder dates according to the prescription date, at least one number of filling days and an allowed filling duration, and the processor further records the filling reminder dates into the historical prescription ledger.

3. The prescription management system of claim 1, wherein the processor further determines a medicine compounding service charge according to a category of the prescription.

4. The prescription management system of claim 1, wherein the storage further stores a medicine recycle ledger and the processor further records the user identity, at least one medicine name included in a medicine recycle item of the user, and a remaining medicine amount corresponding to each of the at least one medicine name into the medicine recycle ledger.

5. The prescription management system of claim 4, wherein the processor further decides a medicine-prescribing prompt by comparing each of the medicine names of the prescription with the at least one medicine name corresponding to the user identity in the medicine recycle ledger.

6. The prescription management system of claim 1, wherein the historical prescription ledger further stores a plurality of prescription records, the processor further retrieves at least one unexpired prescription record of the user from the historical prescription ledger according to the user identity and a use-up date of each of the prescription records, the processor further determines a current medicine ingredient list according to at least one medicine name included in the at least one unexpired prescription record and a medicine ingredient database, the processor further determines a prescription medicine ingredient list according to the at least one medicine name included in the prescription and the medicine ingredient database, and the processor further determines a medicine conflict evaluation result by comparing the current medicine ingredient list and the prescription medicine ingredient list.

7. The prescription management system of claim 6, wherein the storage further stores a medicine rating ledger, the medicine conflict evaluation result is that a first ingredient of a first medicine included in the prescription conflicts with a second ingredient of a second medicine recorded in a specific prescription record among the at least one unexpired prescription record, and the processor further records a physician identity of a physician who prescribes the prescription, the prescription date, the identity code of the prescription, the first medicine, the first ingredient, an identity code of the specific prescription record, the second medicine, and the second ingredient into the medicine rating ledger.

8. The prescription management system of claim 1, wherein the historical prescription ledger further stores a plurality of prescription records, the processor further retrieves at least one unexpired prescription record of the user from the historical prescription ledger according to the user identity and a use-up date of each of the prescription records, the processor further determines a current medicine ingredient list according to at least one medicine name included in the at least one unexpired prescription record and a medicine ingredient database, the processor further determines a prescription medicine ingredient list according to the at least one medicine name included in the prescription and the medicine ingredient database, and the processor further determines a medicine conflict evaluation result according to the current medicine ingredient list, the prescription medicine ingredient list, and a medicine interaction database.

9. The prescription management system of claim 8, wherein the storage further stores a medicine rating ledger, the medicine conflict evaluation result is that a first ingredient of a first medicine included in the prescription conflicts with a second ingredient of a second medicine recorded in a specific prescription record among the at least one unexpired prescription record, and the processor further records a physician identity of a physician who prescribes the prescription, the prescription date, the identity code of the prescription, the first medicine, the first ingredient, an identity code of the specific prescription record, the second medicine, and the second ingredient into the medicine rating ledger.

10. The prescription management system of claim 1, wherein the historical prescription ledger and the insurance payment ledger are stored in the storage individually in the form of a blockchain.

11. A prescription management method, being adapted for a server system, the server system storing a historical prescription ledger and an insurance payment ledger, the prescription management method comprising:
receiving a confirmation signal of prescribing a prescription for a user, wherein the prescription comprises at least one medicine name;
calculating at least one insurance fee and an out-of-pocket fee of the prescription based on the at least one medicine name and a medicine price database;
calculating an expiration date of the prescription according to a prescription date and an allowed number of days of the prescription;
recording an identity code of the prescription, the prescription date, a user identity of the user, the at least one medicine name, the expiration date, the at least one insurance fee, and the out-of-pocket fee of the prescription into the historical prescription ledger; and
recording a prescription filled date, the identity code of the prescription, at least one filled medicine name, the at least one insurance fee, and the out-of-pocket fee into the insurance payment ledger in response to a filling confirmation signal, wherein the at least one filled medicine name corresponds to the at least one medicine name.

12. The prescription management method of claim 11, wherein the prescription is a continuous prescription and the prescription management method further comprises:
calculating a plurality of filling reminder dates according to the prescription date, at least one number of filling days, and an allowed filling duration;
wherein the recording step further records the filling reminder dates into the historical prescription ledger.

13. The prescription management method of claim 11, further comprising:
determining a medicine compounding service charge according to a category of the prescription.

14. The prescription management method of claim 11, wherein the server system further stores a medicine recycle ledger and the prescription management method further comprises:
recording the user identity, at least one medicine name included in a medicine recycle item of the user, and a remaining medicine amount corresponding to each of the at least one medicine name into the medicine recycle ledger.

15. The prescription management method of claim 14, further comprising:
determining a medicine-prescribing prompt by comparing each of the medicine names of the prescription with the at least one medicine name corresponding to the user identity in the medicine recycle ledger.

16. The prescription management method of claim 11, wherein the historical prescription ledger further stores a plurality of prescription records and the prescription management method further comprises:
retrieving at least one unexpired prescription record of the user from the historical prescription ledger according to the user identity and a use-up date of each of the prescription records;
determining a current medicine ingredient list according to at least one medicine name included in the at least one unexpired prescription record and a medicine ingredient database;
determining a prescription medicine ingredient list according to the at least one medicine name included in the prescription and the medicine ingredient database; and
determining a medicine conflict evaluation result by comparing the current medicine ingredient list and the prescription medicine ingredient list.

17. The prescription management method of claim 16, wherein the server system further stores a medicine rating ledger, the medicine conflict evaluation result is that a first ingredient of a first medicine included in the prescription conflicts with a second ingredient of a second medicine recorded in a specific prescription record among the at least one unexpired prescription record, and the prescription management method further comprises:
recording a physician identity of a physician who prescribes the prescription, the prescription date, the identity code of the prescription, the first medicine, the first ingredient, an identity code of the specific prescription record, the second medicine, and the second ingredient into the medicine rating ledger.

18. The prescription management method of claim 11, wherein the historical prescription ledger further stores a plurality of prescription records and the prescription management method further comprises:
retrieving at least one unexpired prescription record of the user from the historical prescription ledger according to the user identity and a use-up date of each of the prescription records;
determining a current medicine ingredient list according to at least one medicine name included in the at least one unexpired prescription record and a medicine ingredient database;
determining a prescription medicine ingredient list according to the at least one medicine name included in the prescription and the medicine ingredient database; and
determining a medicine conflict evaluation result according to the current medicine ingredient list, the prescription medicine ingredient list, and a medicine interaction database.

19. The prescription management method of claim 18, wherein the server system further stores a medicine rating ledger, the medicine conflict evaluation result is that a first ingredient of a first medicine included in the prescription conflicts with a second ingredient of a second medicine recorded in a specific prescription record among the at least one unexpired prescription record, and the prescription management method further comprises:
recording a physician identity of a physician who prescribes the prescription, the prescription date, the identity code of the prescription, the first medicine, the first ingredient, an identity code of the specific prescription record, the second medicine, and the second ingredient into the medicine rating ledger.
